(19) United States
(12) Patent Application Publication
(10) Pub. No.: US 2025/0166197 A1
(43) Pub. Date: May 22, 2025

(54) SYSTEMS AND METHODS FOR DIAGNOSING A SKIN DISEASE

(71) Applicant: Health Strategy, LLC, Peoria, IL (US)

(72) Inventor: Jack Carson, Greenville, SC (US)

(21) Appl. No.: 18/955,750

(22) Filed: Nov. 21, 2024

Related U.S. Application Data
(60) Provisional application No. 63/601,604, filed on Nov. 21, 2023.

(51) Int. Cl. G06T 7/00; A61B 5/00; G06T 7/11

(52) U.S. Cl. CPC G06T 7/0016; A61B 5/445; G06T 7/11; G06T 2207/20084; G06T 2207/30088

(57) ABSTRACT

Disclosed herein are systems and methods for diagnosing skin diseases and/or determining an appropriate treatment. The systems and methods are operable to determine a body surface area of a patient based on one or more images of the patient, apply one or more masks to determine the affected areas of the body surface area, provide a diagnosis based on a calculated percentage of affected areas in relation to the body surface area of the patient, and determine a treatment based on the diagnosis.

### CROSS-REFERENCE TO RELATED APPLICATIONS

This application claims the benefit of U.S. Application No. 63/601,604, filed Nov. 21, 2023, the entire contents of which are incorporated herein by reference in their entirety.

### FIELD OF DISCLOSURE

The present disclosure relates to systems and methods for diagnosing a skin disease and/or determining an appropriate treatment by determining a body surface area of a patient based on one or more images of the patient, segmenting the one or more images into affected areas and unaffected areas, calculating a percentage of the affected areas in relation to the body surface area of the patient, and determining an appropriate treatment based on the diagnosis.

### BACKGROUND

Skin diseases affect a large portion of the worldwide population. In many instances, diagnosis of skin diseases requires multiple visits to a physician's office. Further, to prescribe certain pharmaceuticals extensive paperwork and evaluation by physicians is required.

Therefore, there is a need for automated skin diagnosis and prescription systems and methods.

### SUMMARY

The present disclosure generally relates to a method for diagnosing a skin disease and determining an appropriate treatment by labeling one or more images. The method can include capturing and/or receiving the one or more images of a patient, determining a body surface area of a patient, segmenting the one or more images into affected areas of the body surface area of the patient and unaffected areas of the body surface area of the patient by identifying the affected areas and applying one or more masks to the affected areas, determining a diagnosis from the affected areas of the body surface area, and determining the treatment based on the diagnosis. In an aspect the treatment is determined based on a mild threshold, a moderate threshold, and a severe threshold of the diagnosis.

In various aspects, the determining the diagnosis comprises calculating a percentage of the affected areas in relation to the body surface area of the patient. In an aspect, the mild threshold is greater than 0% of the body surface area of the patient is affected. In an aspect, the moderate threshold is greater than 3% of the body surface area of the patient is affected. In an aspect, the severe threshold is greater than 10% of the body surface area of the patient is affected. In another aspect, determining the body surface area of the patient comprises determining an image scale of the one or more images, a height of the patient, and a weight of the patient. In an aspect, the skin disease is plaque psoriasis. In an aspect, determining the body surface area of the patient, segmenting the one or more images, and determining the diagnosis are conducted by a computing system. In an aspect, a convolutional neural network segments the one or more and determines the diagnosis from the affected areas. In an aspect, the method further includes administering the treatment to the patient. In another aspect, the method further includes repeating the method after a period of time. In an aspect, the period of time is about 1 month to about 12 months. In an aspect, the method further includes determining an efficacy of the treatment based on an improvement in the diagnosis after the period of time.

Further provided herein is a non-transitory computer-readable medium, encoded with instructions for diagnosing and determining a treatment for a skin disease that, when executed by a computing device, cause the computing device to perform operations for diagnosing the skin disease. The operations include receiving one or more images of a patient, determining a body surface area of the patient, segmenting the one or more images into affected areas of the body surface area of the patient and unaffected areas of the body surface of the patient by identifying the affected areas and applying one or more masks to the affected areas, determining a diagnosis from the affected areas of the body surface area, and determining the treatment based on the diagnosis. In an aspect, the treatment is determined based on a mild threshold, a moderate threshold, and a severe threshold of the diagnosis.

In various aspects, determining the diagnosis comprises calculating a percentage of the affected areas in relation to the body surface area of the patient. In an aspect, the mild threshold is greater than 0% of the body surface area of the patient is affected, the moderate threshold is greater than 3% of the body surface area of the patient is affected, and the severe threshold is greater than 10% of the body surface area of the patient is affected. In an aspect, determining the body surface area of the patient comprises determining an image scale of the one or more images, a height of the patient, and a weight of the patient. In an aspect, the skin disease is plaque psoriasis. In an aspect, a convolutional neural network segments the one or more images and determines a diagnosis from the affected areas. In an aspect, the operations further include repeating the operations after a period of time. In an aspect, the period of time is about 1 month to about 12 months. In an aspect, the operations further include determining an efficacy of the treatment based on an improvement in the diagnosis after the period of time.

### BRIEF DESCRIPTION OF FIGURES

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIG. 1 is a flowchart of a method in one example.

FIG. 2 illustrates a graphical user interface in one example.

FIG. 3 illustrates an image and a scale box in one example.

FIG. 4 illustrates a mask box and a mask in one example.

FIG. 5 illustrates an image with a plurality of masks in one example.

FIG. 6 illustrates a plurality of masks in one example.

FIG. 7 illustrates a diagnosis displayed on a graphical user interface in one example.

FIG. 8 illustrates a plurality of masks in one example.

FIG. 9 illustrates a diagnosis displayed on a graphical user interface in one example.

FIG. 10 illustrates a neural network in one example.

FIG. 11 illustrates a convolutional neural network (CNN) in one example.

FIG. 12 illustrates a computing system in one example.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment,” “an embodiment,” or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Provided herein are systems and methods for diagnosing skin diseases. Manual (i.e., visual) diagnosis of skin diseases, and the extent of skin diseases, is time-consuming, expensive, and requires the availability of experienced personnel. To address these issues, automated diagnosis systems and methods have been developed. The systems and methods described herein provide a photo labeling tool that is operable to accurately diagnosis the severity of a skin diseases. The diagnosis can provide efficient and accurate diagnoses which, in turn, can provide efficient, accurate, and appropriate prescriptions to patients affected by skin diseases. Further, the systems and methods can be used to expedite the completion of insurance claims. In an example, the skin disease can be plaque psoriasis or other skin diseases.

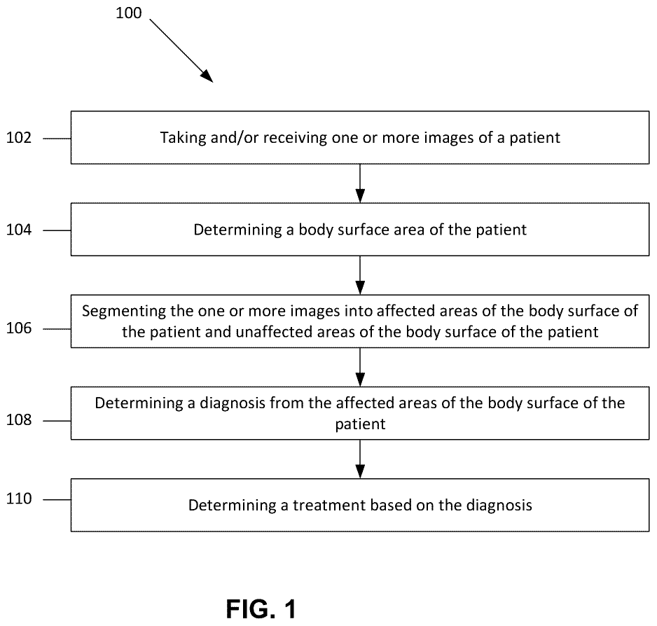

As illustrated in FIG. 1, the method 100 can begin at block 102. At block 102, the method may include receiving one or more images of a patient. The one or more images can include one image to about one hundred images, or more. Each image can display one or more affected areas on a patient's body. The one or more images can include as many images as necessary to capture all the affected areas of the patient's body. The one or more images can be taken using a camera or other image capture device. A patient can take the images themselves, medical staff can take the images, or others can take the images for the patient. Each image can be uploaded to a computing system for diagnosis. In some examples, any image can be used, and the images do not have to be of a standardized format. Once the image is uploaded to a computing system, the image can be embedded based on the properties of the image.

At block 104, the method 100 can include determining a body surface area of the patient based on the patient's height and weight. Equation 1 can be used to calculate the body surface area of the patient.

$${[ \frac{weight ( kg ) \times height ( cm )}{3600} ]}^{0.5} \qquad ( Eq . 1 )$$

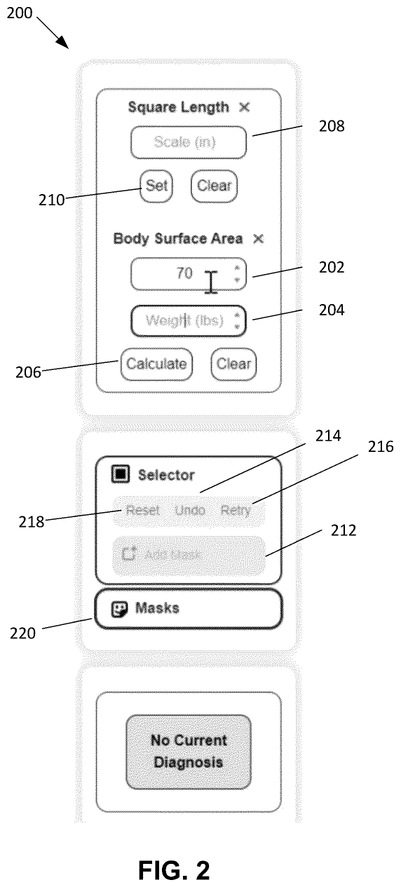

As illustrated in FIG. 2, the method can be conducted on a computing system including a graphical user interface (GUI 200). The GUI 200 can be configured to receive information from an operator. For example, the height of the patient can be input into the height box 202 of the GUI 200 and the weight of the patient can be input into the weight box 204. The operator can then click on the calculate button 206 and the computing system can calculate the body surface area of the patient.

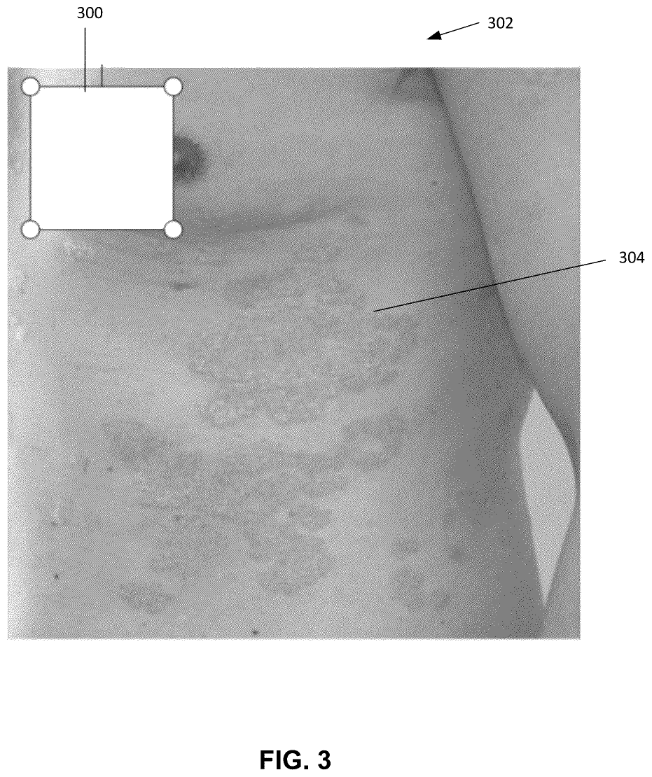

Once the patient's body surface area is determined, the one or more images can be scaled. Scaling the images can comprise indicating a length and/or width of a scale box. In some examples, a physician can put a marking on the patient indicating a known length, the scale box can be fit over the known length, and the known length can be input into the computing system. In this manner, each image can be scaled. As illustrated in FIG. 3, the computing system can provide a scale box 300 overlaid on the image 302. The size of the scale box 300 can be adjusted to have the same length as a known length of a portion of the patient's body. The length of the scale box 300 can be input into the square length box 208 of the GUI 200. The set button 210 can then be clicked. The computing system can then scale the image for diagnosis.

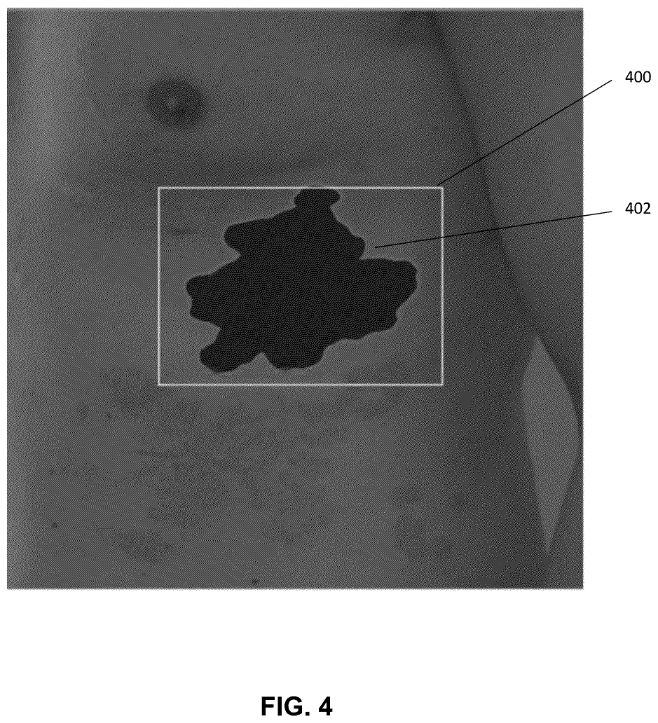

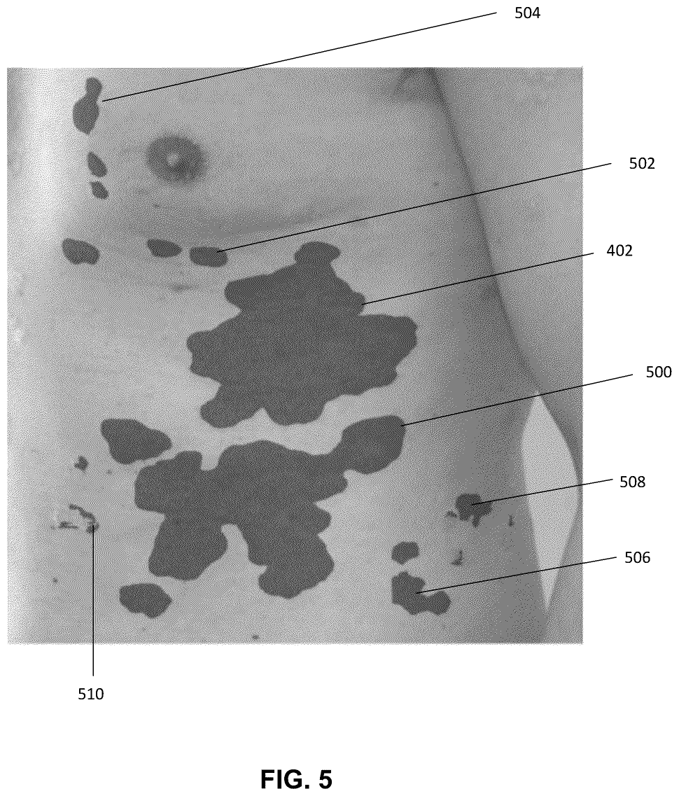

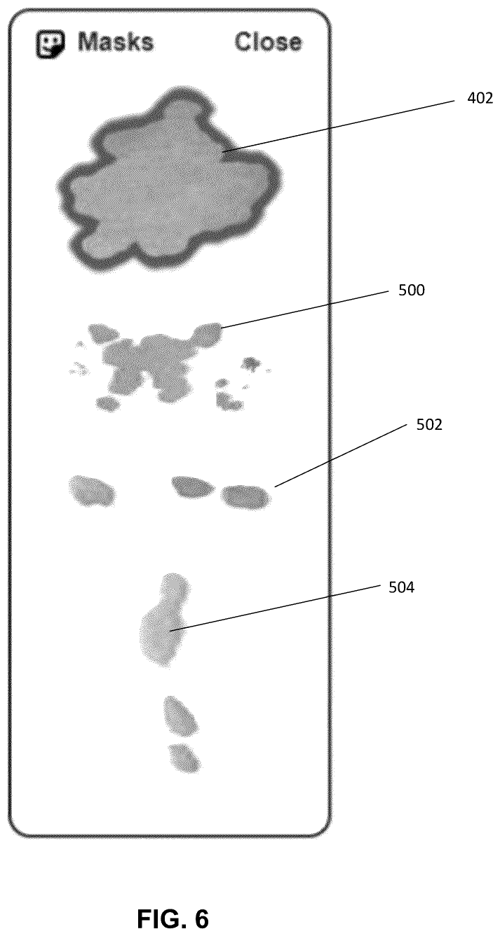

At block 106, the method 100 can include segmenting the one or more images of the patient. Segmenting the one or more images can be accomplished using the computing system. In some examples, the computing system has one or more neural networks, described further herein, that identify and allow for a user to segment images. The neural network can be configured to segment an entire image or user selected areas of an image. Segmenting the one or more images can begin by clicking the add mask 212 button on the GUI 200. As illustrated in FIG. 4, the operator can then drag and fit a box 400 over an affected area 304. The computing system can then determine the differences within the box 400 and segment the affected areas in the box 400, thereby placing a mask 402 on the affected area. The computing system can be configured to place a mask 402 only on the affected area 304 within the box 400. Segmenting the one or more images can include placing more than one mask on the image when there is more than one affected area of the body surface area of the patient. FIG. 5 illustrates multiple masks 402, 500, 502, 504, 506, 508, 510 covering affected areas of the patient's body surface area. When a mask 402, 500, 502, 504, 506, 508, 510 does not correctly cover the proper affected area, the operator can click the undo button 214 or retry button 216 on the GUI 200, to reinput the mask 402, 500, 502, 506, 508, 510. The masks 402, 500, 502, 504, 506, 508, 510 can also be reset by clicking the reset button 218. The operator can click the masks button 220 to view the masks 402, 500, 502, 504 placed on the image, as illustrated in FIG. 6, corresponding to the masks 402, 500, 502, 504 in FIG. 5. The operator can ensure that all of the masks 402, 500, 502, 504, 506, 508, 510 have been properly placed on the image to verify that a correct diagnosis is being made.

The masking process can be repeated for each image. For example, the patient can have multiple areas of their body surface area that are affected by the skin diseases. Multiple images can be taken of the different affected areas and the method 100 can be repeated to determine a total number and surface area of masks for all of the one or more images.

At block 108, the method 100 can include determining a diagnosis from the affected areas of the body surface of the patient. Diagnosing the skin disease can include calculating a percentage of affected areas in relation to the body surface area of the patient. The computing system can calculate a percentage of affected areas by determining a surface area of each mask 402, 500, 502, 504, 506, 508, 510 on the one or more images 302. The surface area of each mask 402 can be added together to determine a total mask surface area (e.g., total affected surface area). The total affected surface area can then be divided by the body surface area of the patient and multiplied by 100 to determine a percentage of the body surface area of the patient that is affected. Equation 2 below illustrates the calculation for a diagnosis.

$$Diagnosis ( \% ) = \frac{total affected surface area}{total body surface area} \times 100 \qquad ( Eq . 2 )$$

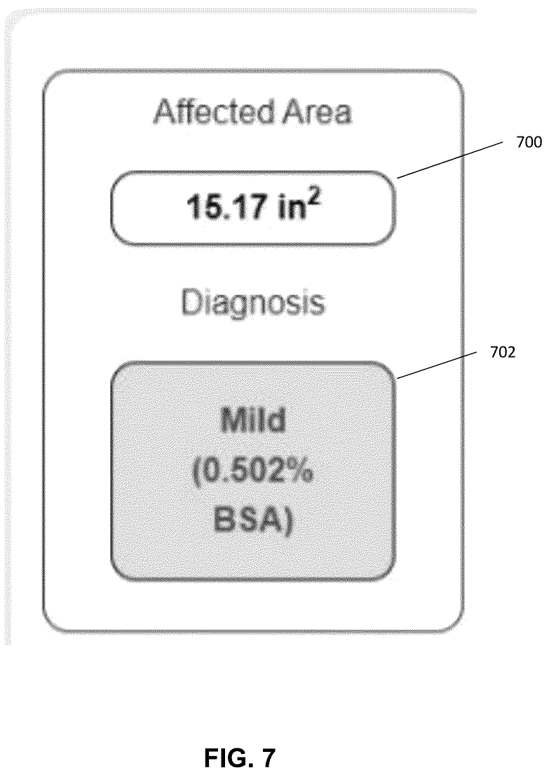

As illustrated in FIG. 7, the computing system can be operable to output the diagnosis 702 and the total affected area 700. The diagnosis can be mild, moderate, or severe. For plaque psoriasis, a mild diagnosis can mean less than 3% of the body surface area of the patient is affected. A moderate diagnosis can mean about 3% to about 10% of the body surface area of the patient is affected. A severe diagnosis can mean more than 10% of the body surface area of the patient is affected. The diagnosis 702 can be provided on a display.

At block 110, the method 100 can further include determining an appropriate treatment based on the diagnosis. The accurate diagnosis provided by the method 100 can be used to accurately prescribe an appropriate treatment to the patient. For example, different pharmaceutical drugs known in the art can be used to treat different diagnoses (e.g., mild, moderate, and severe). The treatment can be based on different diagnoses. For example, the treatment can be based on a mild threshold, a moderate threshold, and a severe threshold. The mild threshold is when greater than 0% of the body surface area of the patient is affected. The moderate threshold is when greater than 3% of the body surface area of the patient is affected. The severe threshold is when greater than 10% of the body surface area of the patient is affected. Using the thresholds, more accurate prescribing of prescription drugs can be accomplished. In some examples, the computing system can output a treatment plan based on the diagnosis. The treatment plan can be provided on a display.

In other examples, a physician or medical personal can provide a treatment plan to the patient based on the diagnosis. In an aspect, the method 100 can further include administering a treatment to the patient based on the diagnosis.

In some aspects, the accuracy of the method 100 can provide detailed diagnoses for tier 1, tier 2, and tier 3 pharmaceutical drugs. In many instances, insurance providers can require detailed evaluation of a skin disease before a tier 3 pharmaceutical drug is covered by the insurance provider. The systems and methods described herein provide accurate and efficient diagnoses which can be used to prescribe tier 3 pharmaceutical drugs. The diagnosis can determine the appropriate treatment. For example, a severe diagnosis can require a more aggressive treatment plan or stronger pharmaceutical drug, while a moderate or mild diagnosis can require a less aggressive treatment plan or pharmaceutical drug.

In another aspect, the method 100 can further include repeating the method 100 after a period of time. The method 100 can be repeated about 1 month to about 2 months, about 2 months to about 3 months, about 3 months to about 4 months, about 4 months to about 5 months, about 5 months to about 6 months, about 6 months to about 7 months, about 7 months to about 8 months, about 8 months to about 9 months, about 9 months to about ten months, about 10 months to about 11 months, about 11 months to about 12 months, or more after the method 100 has been conducted. By repeating the method 100 after the period of time, the efficacy of the prescribed treatment can be determined by determining the improvement or lack thereof in the patient's skin disease. In this manner, the treatment can be adjusted when necessary to provide a more adequate treatment to the patient.

In some aspects, the computing system can include a neural network operable to use machine learning to automatically diagnose images uploaded to the computing system. For example, the neural network can be trained on a plurality of images. The training images (e.g., training data) can be images that were segmented (i.e., masked) using the systems and methods described herein.

In some aspects, the computing system can include a convolutional neural network (CNN) that is operable to segment the one or more images and provide a diagnosis. When a CNN is used, the operator does not need to manually input the masks of the affected areas. The CNN can be configured to automatically mask the affected areas and provide the diagnosis. Using a CNN can greatly reduce the diagnosis time of the systems and methods described herein.

In some aspects, the CNN can also be configured to automatically scale the image and can determine the height, weight, and body surface area of the patient based on the image alone. In this manner, the operator can simply upload one or more images of the affected area and the computing system can output the diagnosis and a recommended treatment without any manual input from the operator.

The CNN can be trained on data collected from previous diagnoses using the systems and methods described herein. For example, the CNN can be trained based on previous segmenting (e.g., masking) of a plurality of images from a plurality of patients. In some examples, the training data can include the height, weight, original image, segmented image, affected area, and diagnosis. The CNN can use the data to segment (i.e., mask) the affected areas and provide the diagnosis.

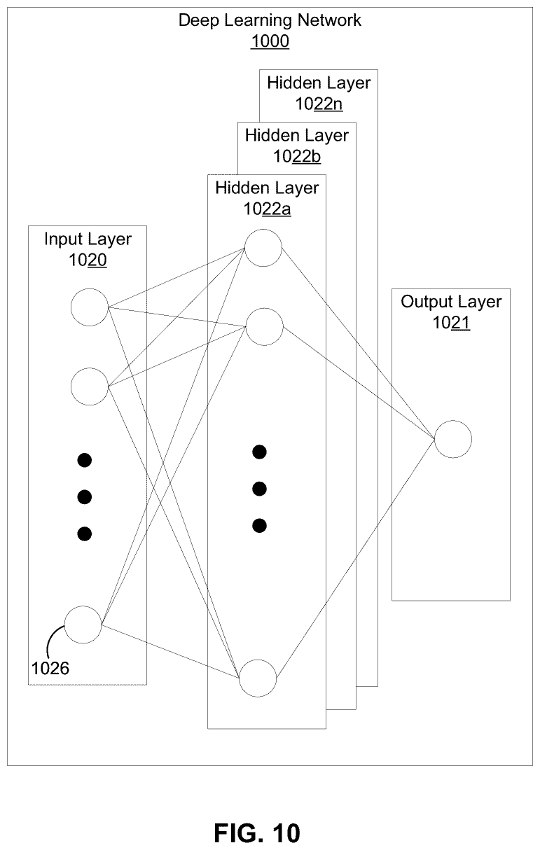

Various aspects of the present disclosure can use machine learning models or systems. FIG. 10 is an illustrative example of a deep learning neural network 1000 that can be used to implement the machine learning-based alignment prediction described herein. An input layer 1020 includes input data. In one illustrative example, the input layer 1020 can include data representing the pixels of an input video frame or image. The neural network 1000 includes multiple hidden layers 1022a, 1022b, through 1022n. The hidden layers 1022a, 1022b, through 1022n include “n” number of hidden layers, where “n” is an integer greater than or equal to one. The number of hidden layers can be made to include as many layers as needed for the given application. The neural network 1000 further includes an output layer 1021 that provides an output resulting from the processing performed by the hidden layers 1022a, 1022b, through 1022n. In one illustrative example, the output layer 621 can provide a classification for an object in an input video frame or image. The classification can include a class identifying the type of object (e.g., affected areas, unaffected areas, total body surface area, etc.).

The neural network 1000 is a multi-layer neural network of interconnected nodes. Each node can represent a piece of information. Information associated with the nodes is shared among the different layers and each layer retains information as information is processed. In some cases, the neural network 1000 can include a feed-forward network, in which case there are no feedback connections where outputs of the network are fed back into itself. In some cases, the neural network 1000 can include a recurrent neural network, which can have loops that allow information to be carried across nodes while reading in input.

Information can be exchanged between nodes through node-to-node interconnections between the various layers. Nodes of the input layer 1020 can activate a set of nodes in the first hidden layer 1022a. For example, as shown, each of the input nodes of the input layer 1020 is connected to each of the nodes of the first hidden layer 1022a. The nodes of the first hidden layer 1022a can transform the information of each input node by applying activation functions to the input node information. The information derived from the transformation can then be passed to and can activate the nodes of the next hidden layer 1022b, which can perform their own designated functions. Example functions include convolutional, up-sampling, data transformation, and/or any other suitable functions. The output of the hidden layer 1022b can then activate nodes of the next hidden layer, and so on. The output of the last hidden layer 1022n can activate one or more nodes of the output layer 1021, at which an output is provided. In some cases, while nodes (e.g., node 1026) in the neural network 1000 are shown as having multiple output lines, a node has a single output and all lines shown as being output from a node represent the same output value.

In some cases, each node or interconnection between nodes can have a weight that is a set of parameters derived from the training of the neural network 1000. Once the neural network 1000 is trained, it can be referred to as a trained neural network, which can be used to classify one or more activities or objects. For example, an interconnection between nodes can represent a piece of information learned about the interconnected nodes. The interconnection can have a tunable numeric weight that can be tuned (e.g., based on a training dataset), allowing the neural network 1000 to be adaptive to inputs and able to learn as more and more data is processed.

The neural network 1000 is pre-trained to process the features from the data in the input layer 1020 using the different hidden layers 1022a, 1022b, through 1022n in order to provide the output through the output layer 1021. In an example in which the neural network 1000 is used to identify features and/or objects in images, the neural network 1000 can be trained using training data that includes both images and labels (e.g., affected areas, unaffected areas, etc.), as described herein. For instance, training images can be input into the network, with each training frame having a label indicating the features in the images (for a feature extraction machine learning system) or a label indicating classes of an activity in each frame. In one example using object classification for illustrative purposes, a training frame can include an image of a number 2, in which case the label for the image can be [0 0 1 0 0 0 0 0 0 0].

In some cases, the neural network 1000 can adjust the weights of the nodes using a training process called backpropagation. As described herein, a backpropagation process can include a forward pass, a loss function, a backward pass, and a weight update. The forward pass, loss function, backward pass, and parameter update is performed for one training iteration. The process can be repeated for a certain number of iterations for each set of training images until the neural network 1000 is trained well enough so that the weights of the layers are accurately tuned.

For the example of identifying features and/or objects in images, the forward pass can include passing a training image through the neural network 1000. The weights are initially randomized before the neural network 1000 is trained. As an illustrative example, a frame can include an array of numbers representing the pixels of the image. Each number in the array can include a value from 0 to 255 describing the pixel intensity at that position in the array. In one example, the array can include a 28×28×3 array of numbers with 28 rows and 28 columns of pixels and 3 color components (such as red, green, and blue, or luma and two chroma components, or the like).

As noted above, for a first training iteration for the neural network 1000, the output will likely include values that do not give preference to any particular class due to the weights being randomly selected at initialization. For example, if the output is a vector with probabilities that the object includes different classes, the probability value for each of the different classes may be equal or at least very similar (e.g., for ten possible classes, each class may have a probability value of 0.1). With the initial weights, the neural network 1000 is unable to determine low level features and thus cannot make an accurate determination of what the classification of the object might be. A loss function can be used to analyze error in the output. Any suitable loss function definition can be used, such as a Cross-Entropy loss. Another example of a loss function includes the mean squared error (MSE).

The loss (or error) will be high for the first training images since the actual values will be much different than the predicted output. The goal of training is to minimize the amount of loss so that the predicted output is the same as the training label. The neural network 1000 can perform a backward pass by determining which inputs (weights) most contributed to the loss of the network and can adjust the weights so that the loss decreases and is eventually minimized. A derivative of the loss with respect to the weights (denoted as dL/dW, where W are the weights at a particular layer) can be computed to determine the weights that contributed most to the loss of the network. After the derivative is computed, a weight update can be performed by updating all the weights of the filters. For example, the weights can be updated so that they change in the opposite direction of the gradient. The weight update can be denoted as w=wᵢ−η*dL/dW, where w denotes a weight, wi denotes the initial weight, and η denotes a learning rate. The learning rate can be set to any suitable value, with a high learning rate including larger weight updates and a lower value indicating smaller weight updates.

The neural network 1000 can include any suitable deep network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. The neural network 1000 can include any other deep network other than a CNN, such as an autoencoder, a deep belief nets (DBNs), a Recurrent Neural Networks (RNNs), BNNs, among others.

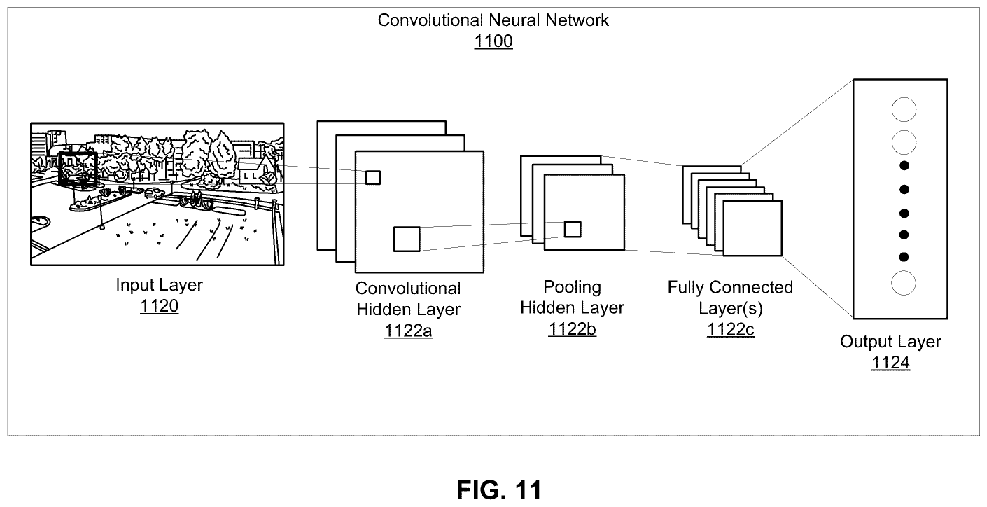

FIG. 11 is an illustrative example of a CNN 1100. The input layer 1120 of the CNN 1100 includes data representing an image or frame. For example, the data can include an array of numbers representing the pixels of the image, with each number in the array including a value from 0 to 255 describing the pixel intensity at that position in the array. Using the previous example from above, the array can include a 28×28×3 array of numbers with 28 rows and 28 columns of pixels and 3 color components (e.g., red, green, and blue, or luma and two chroma components, or the like). The image can be passed through a convolutional hidden layer 1122a, an optional non-linear activation layer, a pooling hidden layer 1122b, and fully connected hidden layers 1122c to get an output at the output layer 1124. While only one of each hidden layer is shown in FIG. 11, one of ordinary skill will appreciate that multiple convolutional hidden layers, non-linear layers, pooling hidden layers, and/or fully connected layers can be included in the CNN 1100. As previously described, the output can indicate a single class of an object or can include a probability of classes that best describe the object in the image.

The first layer of the CNN 1100 is the convolutional hidden layer 1122a. The convolutional hidden layer 1122a analyzes the image data of the input layer 1120. Each node of the convolutional hidden layer 1122a is connected to a region of nodes (pixels) of the input image called a receptive field. The convolutional hidden layer 1122a can be considered as one or more filters (each filter corresponding to a different activation or feature map), with each convolutional iteration of a filter being a node or neuron of the convolutional hidden layer 1122a. For example, the region of the input image that a filter covers at each convolutional iteration would be the receptive field for the filter. In one illustrative example, if the input image includes a 28×28 array, and each filter (and corresponding receptive field) is a 5×5 array, then there will be 24×24 nodes in the convolutional hidden layer 1122a. Each connection between a node and a receptive field for that node learns a weight and, in some cases, an overall bias such that each node learns to analyze its particular local receptive field in the input image. Each node of the hidden layer 1122a will have the same weights and bias (called a shared weight and a shared bias). For example, the filter has an array of weights (numbers) and the same depth as the input. A filter will have a depth of 3 for the video frame example (according to three color components of the input image). An illustrative example size of the filter array is 5×5×3, corresponding to a size of the receptive field of a node.

The convolutional nature of the convolutional hidden layer 1122a is due to each node of the convolutional layer being applied to its corresponding receptive field. For example, a filter of the convolutional hidden layer 1122a can begin in the top-left corner of the input image array and can convolve around the input image. As noted above, each convolutional iteration of the filter can be considered a node or neuron of the convolutional hidden layer 1122a. At each convolutional iteration, the values of the filter are multiplied with a corresponding number of the original pixel values of the image (e.g., the 5×5 filter array is multiplied by a 5×5 array of input pixel values at the top-left corner of the input image array). The multiplications from each convolutional iteration can be summed together to obtain a total sum for that iteration or node. The process is continued at a next location in the input image according to the receptive field of a next node in the convolutional hidden layer 1122a. For example, a filter can be moved by a step amount (referred to as a stride) to the next receptive field. The stride can be set to 1 or another suitable amount. For example, if the stride is set to 1, the filter will be moved to the right by 1 pixel at each convolutional iteration. Processing the filter at each unique location of the input volume produces a number representing the filter results for that location, resulting in a total sum value being determined for each node of the convolutional hidden layer 1122a.

The mapping from the input layer to the convolutional hidden layer 1122a is referred to as an activation map (or feature map). The activation map includes a value for each node representing the filter results at each location of the input volume. The activation map can include an array that includes the various total sum values resulting from each iteration of the filter on the input volume. For example, the activation map will include a 24×24 array if a 5×5 filter is applied to each pixel (a stride of 1) of a 28×28 input image. The convolutional hidden layer 1122a can include several activation maps in order to identify multiple features in an image. The example shown in FIG. 11 includes three activation maps. Using three activation maps, the convolutional hidden layer 1122a can detect three different kinds of features, with each feature being detectable across the entire image.

In some examples, a non-linear hidden layer can be applied after the convolutional hidden layer 1122a. The non-linear layer can be used to introduce non-linearity to a system that has been computing linear operations. One illustrative example of a non-linear layer is a rectified linear unit (ReLU) layer. A ReLU layer can apply the function f(x)=max(0, x) to all of the values in the input volume, which changes all the negative activations to 0. The ReLU can thus increase the non-linear properties of the CNN 1100 without affecting the receptive fields of the convolutional hidden layer 1122a.

The pooling hidden layer 1122b can be applied after the convolutional hidden layer 1122a (and after the non-linear hidden layer when used). The pooling hidden layer 1122b is used to simplify the information in the output from the convolutional hidden layer 1122a. For example, the pooling hidden layer 1122b can take each activation map output from the convolutional hidden layer 1122a and generates a condensed activation map (or feature map) using a pooling function. Max-pooling is one example of a function performed by a pooling hidden layer. Other forms of pooling functions be used by the pooling hidden layer 1122a, such as average pooling, L2-norm pooling, or other suitable pooling functions. A pooling function (e.g., a max-pooling filter, an L2-norm filter, or other suitable pooling filter) is applied to each activation map included in the convolutional hidden layer 1122a. In the example shown in FIG. 11, three pooling filters are used for the three activation maps in the convolutional hidden layer 1122a.

In some examples, max-pooling can be used by applying a max-pooling filter (e.g., having a size of 2×2) with a stride (e.g., equal to a dimension of the filter, such as a stride of 2) to an activation map output from the convolutional hidden layer 1122a. The output from a max-pooling filter includes the maximum number in every sub-region that the filter convolves around. Using a 2×2 filter as an example, each unit in the pooling layer can summarize a region of 2×2 nodes in the previous layer (with each node being a value in the activation map). For example, four values (nodes) in an activation map will be analyzed by a 2×2 max-pooling filter at each iteration of the filter, with the maximum value from the four values being output as the “max” value. If such a max-pooling filter is applied to an activation filter from the convolutional hidden layer 1122a having a dimension of 24×24 nodes, the output from the pooling hidden layer 1122b will be an array of 18×12 nodes.

In some examples, an L2-norm pooling filter could also be used. The L2-norm pooling filter includes computing the square root of the sum of the squares of the values in the 2×2 region (or other suitable region) of an activation map (instead of computing the maximum values as is done in max-pooling) and using the computed values as an output.

Intuitively, the pooling function (e.g., max-pooling, L2-norm pooling, or other pooling function) determines whether a given feature is found anywhere in a region of the image and discards the exact positional information. This can be done without affecting results of the feature detection because, once a feature has been found, the exact location of the feature is not as important as its approximate location relative to other features. Max-pooling (as well as other pooling methods) offer the benefit that there are many fewer pooled features, thus reducing the number of parameters needed in later layers of the CNN 1100.

The final layer of connections in the network is a fully connected layer that connects every node from the pooling hidden layer 1122b to every one of the output nodes in the output layer 1124. Using the example above, the input layer includes 28×28 nodes encoding the pixel intensities of the input image, the convolutional hidden layer 1122a includes 3×24×24 hidden feature nodes based on application of a 5×5 local receptive field (for the filters) to three activation maps, and the pooling hidden layer 1122b includes a layer of 3×12×12 hidden feature nodes based on application of max-pooling filter to 2×2 regions across each of the three feature maps. Extending this example, the output layer 1124 can include ten output nodes. In such an example, every node of the 3×12×12 pooling hidden layer 1122b is connected to every node of the output layer 1124.

The fully connected layer 1122c can obtain the output of the previous pooling hidden layer 1122b (which should represent the activation maps of high-level features) and determines the features that most correlate to a particular class. For example, the fully connected layer 1122c layer can determine the high-level features that most strongly correlate to a particular class and can include weights (nodes) for the high-level features. A product can be computed between the weights of the fully connected layer 1122c and the pooling hidden layer 1122b to obtain probabilities for the different classes. For example, if the CNN 1100 is being used to predict that an object in a video frame is a person, high values will be present in the activation maps that represent high-level features of people (e.g., two legs are present, a face is present at the top of the object, two eyes are present at the top left and top right of the face, a nose is present in the middle of the face, a mouth is present at the bottom of the face, and/or other features common for a person).

In some examples, the output from the output layer 1124 can include an M-dimensional vector (in the prior example, M=10). M indicates the number of classes that the CNN 1100 has to choose from when classifying the object in the image. Other example outputs can also be provided. Each number in the M-dimensional vector can represent the probability the object is of a certain class. In one illustrative example, if a 10-dimensional output vector represents ten different classes of objects is [0 0 0.05 0.8 0 0.15 0 0 0 0], the vector indicates that there is a 5% probability that the image is the third class of object, an 80% probability that the image is the fourth class of object, and a 15% probability that the image is the sixth class of object. The probability for a class can be considered a confidence level that the object is part of that class.

In some examples, the machine learning models and/or neural networks described herein can be trained by a method. The method can be performed based on execution of a training module and a control module by a processor.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The training module can first receive correlation data from the control system. The correlation data can include one or more parameters (e.g., diseased skin, non-diseased skin, body surface area calculations, affected area determinations, etc.) and a correlation coefficient. At a first step, the training module can query a correlation database for correlation data including the one or more parameters and the correlation coefficient. In some examples, the data received from the correlation database can be different than the correlation data received from the control system, such as correlation data previously calculated. In some examples, the correlation data can include the masks placed over affected areas of the patient's skin and/or unmasked portions of the patient's skin.

At a second step, the training module can select correlation data (e.g., masks and/or unmasked areas) from the received correlation data. The selected correlation data includes one or more parameters and a correlation coefficient. At a decision step, the training module can determine whether the selected correlation is above a predetermined correlation threshold. In an ideal environment, the predetermined correlation threshold is 0.90, or 90%, therefore if the correlation coefficient is about 0.90, then the correlation data is above the predetermined correlation threshold. In some examples, the predetermined correlation threshold may be lower, such as 0.70. In some examples, a correlation coefficient which is equal to the predetermined correlation threshold may be considered to be above the predetermined correlation threshold. In an example, the predetermined correlation threshold is 0.90 and the correlation coefficient is 0.92, therefore the correlation data is above the predetermined correlation threshold. In another example, the correlation coefficient is 0.50, and therefore the correlation data is below the predetermined correlation threshold.

At another step, the training module can select correlation data as a feature if the correlation coefficient is above a significance threshold. A feature is measurable property, characteristic, or phenomenon, which can be used to quantify relationships and patterns which can be recognized and utilized by machine-learning models. Features with significant relationships improve the accuracy and relevance of machine-learning models. At a decision step, the training module can determine whether there is more correlation data. There is more correlation data if there are more sets of parameters and correlation coefficients which have not been compared to a significance threshold. If there is more correlation data, then return to the second step and select additional correlation data comprising the one or more parameters and the correlation coefficient. At another step, the training module can query a parameter database for parameter data related to the selected features. For example, the parameter database can include a plurality of skin disorder images with certain characteristics (e.g., features) to determine whether an area of a patient's skin includes a skin disease.

At another step, the training module can train a machine-learning model using the correlation data from the correlation database and the parameter data from the parameter database. Training a machine-learning model may include the steps of providing a set of parameters and/or correlation data to the machine-learning model and receiving at least one prediction from the machine-learning model, where the prediction comprises a value of a parameter for which data is not provided to the machine-learning model. The prediction value is then compared against a known value, and a correction factor is applied based upon the variance of the prediction from the known value. This process may be repeated multiple times for a predetermined number of iterations or until the accuracy of the model is within an acceptable level. In some models, such as using generative adversarial networks, known values may be similarly determined via mathematical simulation.

The trained machine-learning model can be sent to the control system.

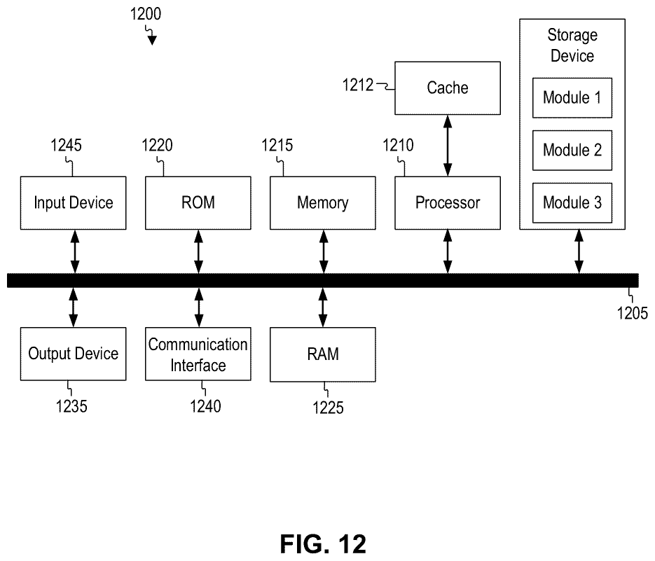

FIG. 12 is a diagram illustrating an example of a system for implementing certain aspects of the present technology. In particular, FIG. 12 illustrates an example of computing system 1200, which can be for example any computing device making up internal computing system, a remote computing system, a camera, or any component thereof in which the components of the system are in communication with each other using connection 1205. Connection 1205 can be a physical connection using a bus, or a direct connection into processor 1210, such as in a chipset architecture. Connection 1205 can also be a virtual connection, networked connection, or logical connection.

In some aspects, computing system 1200 is a distributed system in which the functions described in this disclosure can be distributed within a datacenter, multiple data centers, a peer network, etc. In some aspects, one or more of the described system components represents many such components each performing some or all of the function for which the component is described. In some aspects, the components can be physical or virtual devices.

Example computing system 1200 includes at least one processing unit (CPU or processor) 1210 and connection 1205 that couples various system components including system memory 1215, such as read only memory (ROM) 1220 and read only memory (RAM) 1225 to processor 1210. Computing system 1200 can include a cache 1212 of high-speed memory connected directly with, in close proximity to, or integrated as part of processor 1210.

Processor 1210 can include any general purpose processor and a hardware service or software service, such as services 1232, 1234, and 1236 stored in storage device 1230, configured to control processor 1210 as well as a special-purpose processor where software instructions are incorporated into the actual processor design. Processor 1210 may essentially be a completely self-contained computing system, containing multiple cores or processors, a bus, memory controller, cache, etc. A multi-core processor may be symmetric or asymmetric.

To enable user interaction, computing system 1200 includes an input device 1245, which can represent any number of input mechanisms, such as a microphone for speech, a touch-sensitive screen for gesture or graphical input, keyboard, mouse, motion input, speech, etc. Computing system 1200 can also include output device 1235, which can be one or more of a number of output mechanisms. In some instances, multimodal systems can enable a user to provide multiple types of input/output to communicate with computing system 1200. Computing system 1200 can include communications interface 1240, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Apple® Lightning® port/plug, an Ethernet port/plug, a fiber optic port/plug, a proprietary wired port/plug, a Bluetooth® wireless signal transfer, a BLE wireless signal transfer, an IBEACON® wireless signal transfer, an RFID wireless signal transfer, near-field communications (NFC) wireless signal transfer, dedicated short range communication (DSRC) wireless signal transfer, 802.11 WiFi wireless signal transfer, WLAN signal transfer, Visible Light Communication (VLC), Worldwide Interoperability for Microwave Access (WiMAX), IR communication wireless signal transfer, Public Switched Telephone Network (PSTN) signal transfer, Integrated Services Digital Network (ISDN) signal transfer, 3G/4G/5G/LTE cellular data network wireless signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof. The communications interface 1240 may also include one or more Global Navigation Satellite System (GNSS) receivers or transceivers that are used to determine a location of the computing system 1200 based on receipt of one or more signals from one or more satellites associated with one or more GNSS systems. GNSS systems include, but are not limited to, the US-based GPS, the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 1230 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity module (SIM) card, a mini/micro/nano/pico SIM card, another integrated circuit (IC) chip/card, RAM, static RAM (SRAM), dynamic RAM (DRAM), ROM, programmable read-only memory (PROM), erasable programmable read-only memory (EPROM), electrically erasable programmable read-only memory (EEPROM), flash EPROM (FLASHEPROM), cache memory (L1/L2/L3/L4/L5/L #), resistive random-access memory (RRAM/ReRAM), phase change memory (PCM), spin transfer torque RAM (STT-RAM), another memory chip or cartridge, and/or a combination thereof.

The storage device 1230 can include software services, servers, services, etc., that when the code that defines such software is executed by the processor 1210, it causes the system to perform a function. In some aspects, a hardware service that performs a particular function can include the software component stored in a computer-readable medium in connection with the necessary hardware components, such as processor 1210, connection 1205, output device 1235, etc., to carry out the function. The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as CD or DVD, flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some cases, the computing device or apparatus may include various components, such as one or more input devices, one or more output devices, one or more processors, one or more microprocessors, one or more microcomputers, one or more cameras, one or more sensors, and/or other component(s) that are configured to carry out the steps of processes described herein. In some examples, the computing device may include a display, one or more network interfaces configured to communicate and/or receive the data, any combination thereof, and/or other component(s). The one or more network interfaces can be configured to communicate and/or receive wired and/or wireless data, including data according to the 3G, 4G, 5G, and/or other cellular standard, data according to the Wi-Fi (802.11x) standards, data according to the Bluetooth™ standard, data according to the IP standard, and/or other types of data.

The components of the computing device can be implemented in circuitry. For example, the components can include and/or can be implemented using electronic circuits or other electronic hardware, which can include one or more programmable electronic circuits (e.g., microprocessors, GPUs, DSPs, CPUs, and/or other suitable electronic circuits), and/or can include and/or be implemented using computer software, firmware, or any combination thereof, to perform the various operations described herein.

In some aspects the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

### Examples

In a first example, the systems and methods described herein were used to diagnose plaque psoriasis. First, an image of a patient with plaque psoriasis was taken. The image was then uploaded to the computing system. The height of the patient was 70 inches and the weight of the patient was 170 pounds. This information was input into the graphical user interface and a body surface area of 3,024.91 in² was calculated by the computing system.

Next, the image was scaled. The scale box was used to in conjunction with a known length of a portion of the patient's body. The known length was 5 inches. The length of the scale box was moved by an operator to cover the portion of the body with the known length of 5 inches. The square length of 5 inches was then input by the operator into the square length box of the graphical user interface and the image was scaled.

The operator then began adding masks by clicking the add mask button. Using a mouse, the operator then dragged and fitted the mask box over the affected areas of the body surface of the patient, as illustrated in FIG. 4. The computing system then created a mask of the affected areas within the mask box using a trained neural network. The operator continued adding masks until all of the affected areas were masked, as illustrated in FIG. 5. The masks segmented the image into affected areas and unaffected areas of the body surface.

The computing system then output an affected area of 15.17 in² based on the masks (i.e., segmented image). The computing system also provided a diagnosis. 0.502% of the body surface area was affected by plaque psoriasis and therefore the diagnosis was mild, as illustrated in FIG. 7.

In a second example, the systems and methods described herein were used to diagnose plaque psoriasis of a second patient. First, an image of the second patient with plaque psoriasis was taken. The image was then uploaded to the computing system. The height of the second patient was 70 inches and the weight of the second patient was 160 pounds. This information was input into the graphical user interface and a body surface area of 2934.6 in² was calculated by the computing system.

Next, the image was scaled. The scale box was used in conjunction with a known length of a portion of the patient's body. In this example, the known length of the portion of the patient's body was 5 inches. The length of the scale box was aligned by the user with the known length of the portion of the body part and the set button was clicked. The image was then properly scaled.

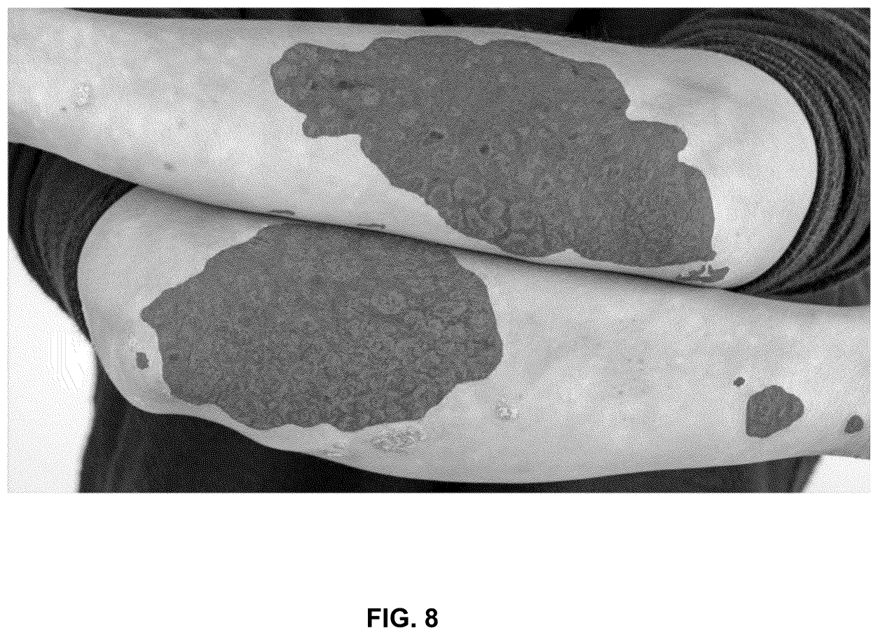

Next, the operator clicked the add mask button. Using a mouse, the operator dragged and fitted the mask box over the affected areas of the body surface of the patient. The computing system then created a mask of the affected areas within the mask box using a trained neural network. The operator continued adding masks until all of the affected areas were masked, as illustrated in FIG. 8. The masks segmented the image into affected areas and unaffected areas of the body surface.

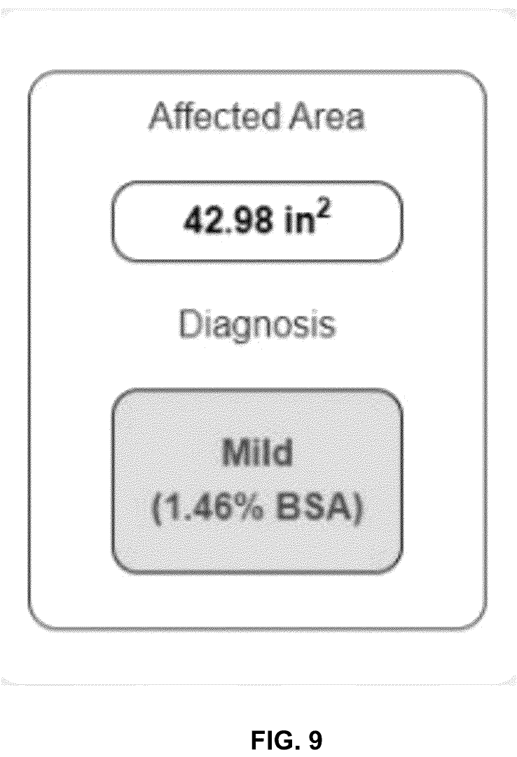

The computing system then provided an affected area of 42.98 in² based on the masks (i.e., segmented image). The computing system also provided a diagnosis. 1.46% of the body surface area was affected by plaque psoriasis and therefore the diagnosis was mild, as illustrated in FIG. 9.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed:

1. A method for diagnosing a skin disease and determining an appropriate treatment by labeling one or more images, the method comprising:
capturing and/or receiving the one or more images of a patient;
determining a body surface area of a patient;
segmenting the one or more images into affected areas of the body surface area of the patient and unaffected areas of the body surface area of the patient by identifying the affected areas and applying one or more masks to the affected areas;
determining a diagnosis from the affected areas of the body surface area; and
determining the treatment based on the diagnosis,
wherein the treatment is determined based on a mild threshold, a moderate threshold, and a severe threshold of the diagnosis.

2. The method of claim 1, wherein determining the diagnosis comprises calculating a percentage of the affected areas in relation to the body surface area of the patient.

3. The method of claim 2, wherein the mild threshold is greater than 0% of the body surface area of the patient is affected, wherein the moderate threshold is greater than 3% of the body surface area of the patient is affected, and wherein the severe threshold is greater than 10% of the body surface area of the patient is affected.

4. The method of claim 1, wherein determining the body surface area of the patient comprises determining an image scale of the one or more images, a height of the patient, and a weight of the patient.

5. The method of claim 1, wherein the skin disease is plaque psoriasis.

6. The method of claim 1, wherein determining the body surface area of the patient, segmenting the one or more images, and determining the diagnosis are conducted by a computing system.

7. The method of claim 6, wherein a convolutional neural network segments the one or more images and determines the diagnosis from the affected areas.

8. The method of claim 1, the method further comprising administering the treatment to the patient.

9. The method of claim 8, the method further comprising repeating the method after a period of time.

10. The method of claim 9, wherein the period of time is about 1 month to about 12 months.

11. The method of claim 10, the method further comprising determining an efficacy of the treatment based on an improvement in the diagnosis after the period of time.

12. A non-transitory computer-readable medium, encoded with instructions for diagnosing and determining an appropriate treatment for a skin disease that, when executed by a computing device, cause the computing device to perform operations for diagnosing the skin disease, the operations comprising:
receiving one or more images of a patient;
determining a body surface area of the patient;
segmenting the one or more images into affected areas of the body surface area of the patient and unaffected areas of the body surface area of the patient by identifying the affected areas and applying one or more masks to the affected areas;
determining a diagnosis from the affected areas of the body surface area; and
determining the treatment based on the diagnosis;
wherein the treatment is determined based on a mild threshold, a moderate threshold, and a severe threshold of the diagnosis.

13. The non-transitory computer-readable medium of claim 12, wherein determining the diagnosis comprises calculating a percentage of the affected areas in relation to the body surface area of the patient.

14. The non-transitory computer-readable medium of claim 13, wherein the mild threshold is greater than 0% of the body surface area of the patient is affected, wherein the moderate threshold is greater than 3% of the body surface area of the patient is affected, and wherein the severe threshold is greater than 10% of the body surface area of the patient is affected.

15. The non-transitory computer-readable medium of claim 12, wherein determining the body surface area of the patient comprises determining an image scale of the one or more images, a height of the patient, and a weight of the patient.

16. The non-transitory computer-readable medium of claim 12, wherein the skin disease is plaque psoriasis.

17. The non-transitory computer-readable medium of claim 12, wherein a convolutional neural network segments the one or more images and determines the diagnosis from the affected areas.

18. The non-transitory computer-readable medium of claim 12, the operations further comprising repeating the operations after a period of time.

19. The non-transitory computer-readable medium of claim 18, wherein the period of time is about 1 month to about 12 months.

20. The non-transitory computer-readable medium of claim 19, the operations further comprising determining an efficacy of the treatment based on an improvement in the diagnosis after the treatment has been administered for the period of time.
